(19) United States
(12) Patent Application Publication
(10) Pub. No.: US 2026/0317730 A1
(43) Pub. Date: Oct. 15, 2026

(54) METHODS FOR PRODUCING POLYBACTERIOPHAGE COMPOSITIONS WITH ENHANCED STABILITY AND LYTIC ACTIVITY FOR PHARMACEUTICAL USE

(71) Applicants: Mirkamol Mirkabilovich MIRSALIYEV, Tashkent (UZ); Shukurulla Raxmatullayevich KASIMOV, Tashkent (UZ); Ergash Turgunovich KADIROV, Tashkent (UZ)

(72) Inventors: Mirkamol Mirkabilovich MIRSALIYEV, Tashkent (UZ); Shukurulla Raxmatullayevich KASIMOV, Tashkent (UZ); Ergash Turgunovich KADIROV, Tashkent (UZ)

(21) Appl. No.: 19/466,303

(22) Filed: Jan. 31, 2026

(30) Foreign Application Priority Data
Feb. 15, 2025 (UZ) FAP 20250053

(51) Int. Cl. C12N 7/02; A61K 35/76; A61K 47/22; C12N 1/06; C12N 1/20; C12R 1/01

(52) U.S. Cl. CPC C12N 7/025; A61K 35/76; A61K 47/22; C12N 1/06; C12N 1/20; C12N 2795/00032; C12N 2795/00052; C12R 2001/01

(57) ABSTRACT

Methods are provided for producing polybacteriophage compositions suitable for pharmaceutical use. Bacterial host strains selected from Staphylococcus haemolyticus, Pseudomonas aeruginosa, Proteus mirabilis, Klebsiella pneumoniae, Enterococcus faecalis, and Escherichia coli are separately cultivated in soy broth with casein digest, expanded to high titers, and inoculated with corresponding seed bacteriophages under controlled aeration to obtain phagolysates. The phagolysates are combined in equal proportions, stabilized with quinosol (8-hydroxyquinoline sulfate) preservative at 0.001% (w/v), and sterilized by cascade membrane filtration (1.2 μm to 0.2 μm). The resulting polybacteriophage composition is sterile, non-toxic, stable during storage, and exhibits high lytic activity (Gracia titer 10⁻⁶ to 10⁻⁸) against multiple pathogenic bacterial species. Pharmaceutical compositions and methods of treating bacterial infections are also provided.

### CROSS-REFERENCE TO RELATED APPLICATIONS

This application claims priority under 35 U.S.C. § 119(a) to Uzbekistan Utility Model Patent No. FAP 2798, Application No. FAP 20250053, filed Feb. 15, 2025, and granted Aug. 25, 2025, entitled “Polibakteriofagni olish usuli” (“Method for Obtaining a Polybacteriophage”), the entire contents of which are incorporated herein by reference in their entirety.

The priority document has been assigned International Patent Classification C12N7/00 (2006.01), which relates to viruses, e.g. bacteriophages; compositions thereof; preparation or purification thereof. The present application is entitled to the priority date of Feb. 15, 2025, in accordance with the Paris Convention for the Protection of Industrial Property and 35 U.S.C. § 119(a).

### TECHNICAL FIELD OF THE INVENTION

The present invention relates generally to the pharmaceutical and biotechnological industries. More particularly, the invention relates to methods for producing sterile, stable, multi-component polybacteriophage compositions having high specific lytic activity against multiple clinically relevant pathogenic bacterial species including Staphylococcus haemolyticus, Pseudomonas aeruginosa, Proteus mirabilis, Klebsiella pneumoniae, Enterococcus faecalis, and Escherichia coli. The invention further relates to pharmaceutical compositions comprising such polybacteriophages and to methods for their therapeutic and prophylactic use in treating bacterial infections, including infections caused by multidrug-resistant organisms.

### BACKGROUND OF THE INVENTION

Bacteriophages (phages) are viruses that selectively infect and lyse bacterial cells and represent one of the most abundant biological entities on Earth. First discovered independently by Frederick Twort in 1915 and Felix d'Herelle in 1917, bacteriophages have been studied for over a century as potential therapeutic agents against bacterial infections. The term “bacteriophage” derives from the Greek “bacteria” and “phagein” (to devour), reflecting their ability to destroy bacterial cells through a lytic cycle.

Polybacteriophage preparations, which comprise multiple bacteriophages active against different bacterial species or strains, are particularly valuable for treating polymicrobial infections and infections caused by antibiotic-resistant organisms. Such preparations extend the therapeutic coverage of single-phage preparations while maintaining the specificity advantages inherent to bacteriophage therapy.

The emergence and rapid global spread of antibiotic-resistant bacteria has created an urgent and critical need for alternative antimicrobial therapies. The World Health Organization has identified antimicrobial resistance as one of the top ten global public health threats facing humanity. Particularly concerning are infections caused by multidrug-resistant pathogens, which are responsible for the majority of nosocomial infections.

Bacteriophage therapy offers several significant advantages over conventional antibiotics, including: (a) high specificity for target bacteria, resulting in minimal disruption of commensal microbiota; (b) self-amplification at sites of infection; (c) natural co-evolution with bacterial hosts; (d) ability to disrupt bacterial biofilms; and (e) favorable safety profile with minimal adverse effects.

However, large-scale pharmaceutical production of polybacteriophages presents significant technical challenges including inconsistent bacterial host growth, variable bacteriophage yields, contamination risks, filtration difficulties caused by high-molecular-weight media components, and product instability during storage.

Russian Patent No. RU 2,036,232 describes a method for producing a polybacteriophage by combining bacteriophages obtained after cultivation in liquid nutrient medium with bacteria and phages. However, this reference employs nutrient media such as Martin broth, Hottinger broth, meat-peptone broth, or broth based on enzymatic hemohydrolysate. These media are non-standardized in composition and contain excessive high-molecular-weight proteins and peptides that complicate sterilizing filtration, reduce product yield, and adversely affect pharmaceutical purity.

Russian Patent No. RU 2,153,534 describes additional methods for polybacteriophage production but similarly relies on conventional nutrient media with the attendant disadvantages.

Russian Patent No. RU 2,247,151 describes a method for producing a polybacteriophage by combining multiple bacteriophages obtained by cultivation in a casein-acid nutrient medium with sterilizing filtration through membranes having a pore size of 0.2 μm. A significant disadvantage of this reference is the absence of preservatives in the preparation that would stabilize microbiological purity during production, storage, and use. Without a preservative, contamination may occur, leading to decreased specific lytic and antibacterial activity of the bacteriophages.

Accordingly, there exists a need in the art for improved methods for producing polybacteriophage compositions that provide: (i) reproducible and optimal bacterial host strain growth using a single standardized medium; (ii) efficient bacteriophage propagation with consistently high lytic titers; (iii) effective stabilization to maintain microbiological purity; (iv) reliable sterilization without loss of phage activity; and (v) consistent high lytic activity throughout the product shelf life.

### SUMMARY OF THE INVENTION

The present invention addresses the deficiencies of the prior art by providing novel methods for producing polybacteriophage compositions and pharmaceutical compositions produced thereby. The invention is based on the discovery that the use of soy broth with casein digest as a universal nutrient medium provides superior and uniform growth of multiple bacterial host strains compared to conventional media, achieving bacterial cell yields of 2.0-2.5 times higher than prior art media. The invention further demonstrates that the addition of 8-hydroxyquinoline sulfate (quinosol) as a preservative effectively maintains microbiological purity without adversely affecting bacteriophage activity.

In one aspect, the invention provides a method for producing a polybacteriophage composition comprising: (a) separately cultivating bacterial host strains in a liquid nutrient medium comprising soy broth with casein digest; (b) expanding each bacterial host strain to obtain a bacterial culture having a titer of at least about 10¹ CFU/mL; (c) inoculating each expanded bacterial culture with a corresponding seed bacteriophage under controlled aeration conditions to obtain a phagolysate; (d) combining the phagolysates into a single polybacteriophage preparation; (e) adding a preservative comprising quinosol to the combined phagolysates; and (f) sterilizing the polybacteriophage preparation by cascade membrane filtration.

In another aspect, the invention provides a pharmaceutical polybacteriophage composition comprising bacteriophages active against multiple bacterial species and a pharmaceutically acceptable preservative, wherein the composition is sterile, non-toxic, and exhibits lytic activity of at least about 10⁻⁶ to 10⁻⁸ as measured by the Gracia method.

In yet another aspect, the invention provides methods of treating or preventing bacterial infections in a subject comprising administering to the subject a therapeutically effective amount of a polybacteriophage composition of the invention.

### BRIEF DESCRIPTION OF THE DRAWINGS

The invention will be more fully understood from the following detailed description taken in conjunction with the accompanying drawings, in which:

FIG. 1 is a schematic process flow diagram illustrating an exemplary method for producing a polybacteriophage composition according to the invention, showing the sequential steps of separate bacterial culture propagation, bacteriophage inoculation and amplification, phagolysate recovery, combination of multiple phagolysates, addition of preservative (quinosol), and sterilizing filtration to produce the final pharmaceutical composition.

FIG. 2 is a schematic diagram illustrating an exemplary bioreactor configuration for the bacteriophage propagation stage of the invention, showing the principal components including the culture vessel, temperature control system, aeration system, agitation mechanism, and harvest outflow for phagolysate recovery.

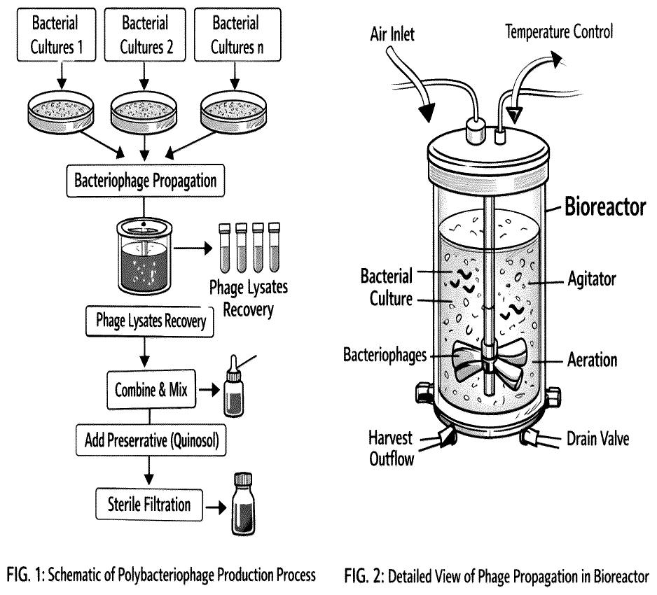

### DETAILED DESCRIPTION OF THE INVENTION

Unless defined otherwise, all technical and scientific terms used herein have the same meaning as commonly understood by one of ordinary skill in the art to which this invention belongs.

As used herein, “bacteriophage” or “phage” refers to a virus that infects and replicates within bacteria. Bacteriophages are composed of proteins that encapsulate a DNA or RNA genome and replicate within the bacterium following injection of their genetic material into the bacterial cytoplasm.

As used herein, “polybacteriophage” refers to a composition comprising two or more bacteriophages, each active against a different bacterial species or strain.

As used herein, “phagolysate” refers to the liquid product obtained following bacteriophage-mediated lysis of bacterial host cells, containing progeny bacteriophage particles, bacterial cell debris, and residual culture medium.

As used herein, “host strain” or “producer strain” refers to a bacterial strain that supports the replication of a specific bacteriophage.

As used herein, “seed bacteriophage” or “mother bacteriophage” refers to a characterized stock preparation of bacteriophage used to initiate infection of bacterial host cultures.

As used herein, “Gracia titer” refers to the bacteriophage titer as measured by the Gracia method (double agar overlay method), expressed as the reciprocal of the highest dilution showing complete bacterial lysis.

As used herein, “CFU/mL” refers to colony-forming units per milliliter, a standard measure of viable bacterial cell concentration.

As used herein, “PFU/mL” refers to plaque-forming units per milliliter, a standard measure of infectious bacteriophage particle concentration.

As used herein, “quinosol” refers to 8-hydroxyquinoline sulfate, a preservative compound with antimicrobial properties.

As used herein, “soy broth with casein digest” refers to a liquid nutrient medium comprising enzymatically digested soybean protein and enzymatically digested casein.

The terms “about” or “approximately” when used in conjunction with a numerical value refer to a range of 10% of the stated value.

### Bacterial Host Strains

The methods of the invention employ separate cultivation of bacterial host strains to produce bacteriophages active against clinically important pathogenic bacteria. In preferred embodiments, the host strains are:
Staphylococcus haemolyticus No. 353/47, Registration No. 007513
Pseudomonas aeruginosa No. 1, Registration No. 005087
Proteus mirabilis No. 0616, Registration No. 007477
Klebsiella pneumoniae No. 0050, Registration No. 007455
Enterococcus faecalis No. 350, Registration No. 007494
Escherichia coli No. 153, Registration No. 007483

These strains are verified, tested strains with typical morphological, biochemical, and serological properties, deposited in the National Collection of Microorganisms of the Center for Epidemiology, Microbiology of Infectious and Parasitic Diseases of the Ministry of Health of the Republic of Uzbekistan.

### Seed Bacteriophages

The methods of the invention employ characterized seed bacteriophages specific for each bacterial host strain:
Staphylococcus haemolyticus bacteriophage No. BSHM No. 2022, Registration No. 007533
Pseudomonas aeruginosa bacteriophage No. BPsAM No. 2019, Registration No. 007534
Proteus mirabilis bacteriophage No. BPrM No. 2019, Registration No. 007532
Klebsiella pneumoniae bacteriophage No. BKPM No. 2022, Registration No. 007535
Enterococcus faecalis bacteriophage No. BEnFM No. 2022, Registration No. 007541
Escherichia coli bacteriophage No. BEsCM No. 2020, Registration No. 007542

### Nutrient Medium

A critical aspect of the invention is the use of soy broth with casein digest as the universal nutrient medium throughout cultivation. This medium provides: (a) universal suitability for all six bacterial host strains; (b) balanced nutrition rich in amino acids and peptides; (c) reduced ballast substances facilitating filtration; (d) standardized composition for batch consistency; and (e) superior yield of 2.0-2.5 times higher than prior art media.

### Process Overview

Stage I—Initial Cultivation: A bacterial culture of each host strain is grown separately at 35-37° C. for 18-24 hours in a test tube containing 5 mL of liquid nutrient medium.
Stage II—Colony Isolation: Bacterial cultures are streaked onto Petri dishes containing solid nutrient medium (20 mL) to obtain isolated single colonies. Incubation at 35-37° C. for 18-24 hours.
Stage III—Culture Expansion: Single colonies are expanded in flasks (500 mL capacity, 250 mL medium) then transferred to culture flasks (1500 mL capacity, 600 mL medium) and inoculated with 40 mL bacterial culture at 10⁹ CFU/mL. After 24 hours, cultures achieve 2.0-2.5×10⁹ CFU/mL.
Stage IV—Phage Propagation: Bioreactors (70 L capacity) containing 50 L of sterilized medium (110° C., 40 min) are inoculated at 37° C. with bacterial culture at 90-100×10⁶ CFU/mL and seed bacteriophage at 10⁴ PFU/mL (0.5% v/v). Cultures are maintained for 6-18 hours with continuous stirring and aeration at 1.2 L air/L medium min until complete lysis. Phagolysates (50 L each) are recovered.
Stage V—Combination and Stabilization: The six phagolysates are combined in equal proportions (1:1:1:1:1:1). A 0.001% (w/v) solution of quinosol is added and the mixture is incubated for 15 minutes.
Stage VI—Sterilizing Filtration: Cascade filtration through membranes with pore sizes from 1.2 μm to 0.2 μm yields a sterile, transparent, light yellow polybacteriophage composition with Gracia titer of 10⁻⁶ to 10⁻⁸.

### Comparative Studies

Extensive experimental studies compared bacterial growth yields in different nutrient media. Table 1 presents comparative data on microbial cell yields (billions per mL) in three different nutrient media.

TABLE 1
Comparative Bacterial Growth Yields in Different Nutrient Media

| | Casein-Acid | Meat-Peptone | Soy Broth with |
|---|---|---|---|
| Host Strain | Medium | Broth | Casein Digest |
| S. haemolyticus No. | 1.5-1.8 | 1.4-1.85 | 2.0-2.49 |
| 353/47 | | | |
| P. aeruginosa No. 1 | 1.6-1.75 | 1.6-1.79 | 2.0-2.50 |
| P. mirabilis No. 0616 | 1.89-1.92 | 1.84-1.89 | 2.0-2.49 |
| K. pneumoniae No. 0050 | 1.78-1.81 | 1.85-1.87 | 2.0-2.50 |
| E. faecalis No. 350 | 1.56-1.73 | 1.74-1.77 | 2.0-2.50 |
| E. coli No. 153 | 1.89-1.91 | 1.85-1.89 | 2.0-2.50 |

Values represent billions of CFU/mL (10⁹ CFU/mL) grown after 18-24 hours incubation at 35-37° C. from an initial inoculum of 1.0 × 10⁹ CFU/mL.

### EQUIVALENTS

Those skilled in the art will recognize, or be able to ascertain using no more than routine experimentation, many equivalents to the specific embodiments of the invention described herein. Such equivalents are intended to be encompassed by the following claims. All patents, patent applications, and publications cited herein are hereby incorporated by reference in their entirety.

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

### Biological Deposit Information

The following biological materials have been deposited with the National Collection of Microorganisms of the Center for Epidemiology, Microbiology of Infectious and Parasitic Diseases of the Ministry of Health of the Republic of Uzbekistan:

### Host Strains (Producer Strains):

Staphylococcus haemolyticus No. 353/47, Registration No. 007513
Pseudomonas aeruginosa No. 1, Registration No. 005087
Proteus mirabilis No. 0616, Registration No. 007477
Klebsiella pneumoniae No. 0050, Registration No. 007455
Enterococcus faecalis No. 350, Registration No. 007494
Escherichia coli No. 153, Registration No. 007483

### Seed Bacteriophages (Monophages):

Staphylococcus haemolyticus bacteriophage No. BSHM No. 2022, Registration No. 007533
Pseudomonas aeruginosa bacteriophage No. BPsAM No. 2019, Registration No. 007534
Proteus mirabilis bacteriophage No. BPrM No. 2019, Registration No. 007532
Klebsiella pneumoniae bacteriophage No. BKPM No. 2022, Registration No. 007535
Enterococcus faecalis bacteriophage No. BEnFM No. 2022, Registration No. 007541
Escherichia coli bacteriophage No. BEsCM No. 2020, Registration No. 007542

Certificates of deposit for all biological materials are maintained with the application records and will be made available in accordance with applicable patent regulations upon patent grant.

What is claimed is:

1. A method for producing a polybacteriophage composition, comprising:
(a) separately cultivating a plurality of bacterial host strains in a liquid nutrient medium comprising soy broth with casein digest, wherein the plurality of bacterial host strains comprises at least two strains selected from the group consisting of Staphylococcus haemolyticus, Pseudomonas aeruginosa, Proteus mirabilis, Klebsiella pneumoniae, Enterococcus faecalis, and Escherichia coli;
(b) expanding each bacterial host strain to obtain a bacterial culture having a titer of at least about 10¹ CFU/mL;
(c) inoculating each expanded bacterial culture with a corresponding seed bacteriophage to obtain a phagolysate;
(d) combining the phagolysates into a single polybacteriophage preparation;
(e) adding a preservative to the combined phagolysates; and
(f) sterilizing the polybacteriophage preparation by membrane filtration through at least one membrane having a pore size of about 0.2 μm.

2. The method of claim 1, wherein the plurality of bacterial host strains comprises Staphylococcus haemolyticus, Pseudomonas aeruginosa, Proteus mirabilis, Klebsiella pneumoniae, Enterococcus faecalis, and Escherichia coli.

3. The method of claim 1, wherein the preservative comprises 8-hydroxyquinoline sulfate (quinosol).

4. The method of claim 3, wherein the quinosol is present at a concentration of approximately 0.001% (w/v).

5. The method of claim 1, wherein the inoculating step comprises adding the seed bacteriophage at a titer of approximately 10⁴ PFU/mL.

6. The method of claim 1, wherein the inoculating step comprises adding the seed bacteriophage in an amount of approximately 0.5% of the volume of the nutrient medium.

7. The method of claim 1, wherein the cultivating step is performed at a temperature of approximately 35-37° C.

8. The method of claim 1, wherein the inoculating step is performed at a temperature of approximately 37° C. under continuous stirring with aeration at an intensity of approximately 1.2 L air/L medium min.

9. The method of claim 1, wherein the expanding step comprises achieving a bacterial culture titer of approximately 2.0-2.5×10⁹ CFU/mL.

10. The method of claim 1, wherein the phagolysates are combined in substantially equal proportions.

11. The method of claim 1, wherein the sterilizing step comprises filtration through a cascade of filters having pore sizes ranging from approximately 1.2 μm to 0.2 μm.

12. The method of claim 1, wherein the liquid nutrient medium is substantially free of meat-peptone broth, Martin broth, Hottinger broth, and hemohydrolysate.

13. The method of claim 1, wherein the polybacteriophage composition has a Gracia titer of at least about 10⁻⁶ to 108 for each monobacteriophage component.

14. The method of claim 1, wherein the inoculating step is performed with a bacterial culture at a concentration of approximately 90-100×10⁶ CFU/mL per 1 mL of nutrient medium.

15. A polybacteriophage composition produced by the method of claim 1.

16. A pharmaceutical polybacteriophage composition comprising:
(a) bacteriophages active against at least two bacterial species selected from the group consisting of Staphylococcus haemolyticus, Pseudomonas aeruginosa, Proteus mirabilis, Klebsiella pneumoniae, Enterococcus faecalis, and Escherichia coli; and
(b) a pharmaceutically acceptable preservative;
wherein the composition is sterile and exhibits lytic activity of at least about 10⁻⁶ as measured by the Gracia method.

17. The composition of claim 16, wherein the preservative comprises quinosol at a concentration of approximately 0.001% (w/v).

18. The composition of claim 16, wherein the composition has a pH of approximately 6.7 to 7.0.

19. The composition of claim 16, wherein the composition is non-toxic and stable during storage.

20. A method of treating or preventing a bacterial infection in a subject in need thereof, comprising administering to the subject a therapeutically effective amount of the polybacteriophage composition of claim 15.
